(19) United States
(12) Patent Application Publication
(10) Pub. No.: US 2004/0171010 A1
(43) Pub. Date: Sep. 2, 2004

(54) Methods

(76) Inventor: Rachael Moore, Cheshire (GB)

Correspondence Address: Fish & Richardson, 225 Franklin Street, Boston, MA, 02110, US

(21) Appl. No.: 10/478,267

(22) PCT Filed: May 15, 2002
(86) PCT No.: PCT/GB02/02294

Related U.S. Application Data
(60) Provisional application No. 60/292,926, filed on May 24, 2001.

(51) Int. Cl. C12Q 1/68

(52) U.S. Cl. 435/6

(57) ABSTRACT

The invention provides a method for the detection of a polymorphism in OATPB in a human which method comprises determining the sequence of the human at one of the following positions: position (1113) of SEQ ID NO: 4 and/or position (312) of SEQ ID NO: 5. The polymorphism at position (1113) is presence of G and/or A and the polymorphism at position (312) is presence of Arg and/or Gln. The method for detection of a nucleic acid polymorphism is selected from amplification refractory mutation system and restriction fragment length polymorphism. The invention also provides use of the method to assess the pharmacogenetics of a drug transportable by OATB.

### METHODS

This invention relates to polymorphism in the human OATPB gene and corresponding novel allelic polypeptides encoded thereby. The invention also relates to methods and materials for analysing allelic variation in the OATPB gene, and to the use of OATPB polymorphism in treatment of diseases with OATPB transportable drugs.

The human sodium independent organic anion transporting polypeptide (OATP) B gene is a member of the OATP supergene family involved in multifunctional transport of organic anions¹. There is an alternative nomenclature for this family as SLC21A (solute carriers) and OATPB relates to SLC21A9. OATPB has a 35% identity at the amino acid level with its gene family member human OATPC (SLC21A6)². OATPC has been shown to be involved in the transport of drugs involved in lipid lowering e.g. statins. Statins have been referred to as a first-line therapy for patients with atherosclerotic vascular diseases³. OATPB has been shown to transport similar substrates as OATPC such as bromosulphophthalcin (BSP) and sulfated steroids (e.g. dehydroepiandrosterone sulfate (DHEAS) and estrone-3-sulphate), but not bile salts. OATPB has a wide tissue expression pattern, with strong expression in the liver and has been localised to the basolateral membranes of human hepatocytes⁴. ¹Biochemical and Biophysical Research Communications 273, 251-260 (2000) Molecular Identification and Characterization of novel members of the Human Organic Anion Transporter (OATP) Family Ikumi Tamai, Jun-ichi Nezu, Hiroshi Uchino, Yoshimichi Sai, Asuka Oku, Miyuki Shimane, Akira Tsuji ²Gasteroenterology 2001 120: 525-533 Organic Anion-Transporting Polypeptide B (OATP-B) and Its Functional Comparison with three other OATPs of human liver. Gerd A Kullak-Ublick, Manfred G. Ismair, Bruno Stieger, Lukas Landmann, Robert Huber, Falvia Pizzagalli, Flavia Pizzagalli, Peter J Meier and Bruno Hagenbuch ³J. Biol Chem 274, 37161-37168 (1999) A Novel Human Hepatic Organic Anion Transporting Polypeptide (OATP2). Identification of a liver-specific human organic anion transporting polypeptide and identification of rat and human hydroxymethylglutaryl-CoA reductase inhibitor transporters. Bonnie Hsiang, Yingjie Zhu, Zhaoqing Wang, Yuli Wu, Vito Sasseville, Wen-Pin Yang, and Todd G. Kirchgessner ⁴Hepatology (Abstract ) October 2000 305A Characterization of OATP-B as a further human liver organic anion transporting polypeptide and its comparison with OATP-A and OATP-C Gerd A Kullak-Ublick, Karin E Fattinger, Bruno Stieger, Lukas Landmann, Robert Huber, Falvia Pizzagalli, Peter J Meier, Bruno Hagenbuch.

All positions herein of polymorphisms in the OATPB polynucleotide relate to the position in SEQ ID NO 4 unless stated otherwise or apparent from the context.

All positions herein of polymorphisms in the OATPB polypeptide relate to the position in SEQ ID NO 5 unless stated otherwise or apparent from the context.

One approach is to use knowledge of polymorphisms to help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Clinical trials have shown that patient response to treatment with pharmaceuticals is often heterogeneous. Thus there is a need for improved approaches to pharmaceutical agent design and therapy.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

The present invention is based on the discovery of a polymorphism in OATPB.

According to one aspect of the present invention there is provided a method for the detection of a polymorphism in OATPB in a human, which method comprises determining the sequence of the human at one of the following positions: position 1113 of SEQ ID NO: 4 and/or position 312 of SEQ ID NO 5.

The term human includes both a human having or suspected of having a OATPB mediated response to a drug and an asymptomatic human who may be tested for predisposition or susceptibility to such a response. At each position the human may be homozygous for an allele or the human may be a heterozygote.

In one embodiment of the invention preferably the polymorphism at position 1113 is presence of G and/or A.

In one embodiment of the invention preferably the polymorphism at position 312 is presence of Arg and/or Gln.

Preferred methods for detection of nucleic acid polymorphism are amplification refractory mutation system and restriction fragment length polymorphism.

The test sample of nucleic acid is conveniently a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| bp | base pair |
| CMC | Chemical mismatch cleavage |
| COPS | Competitive oligonucleotide priming system |
| DGGE | Denaturing gradient gel electrophoresis |
| FRET | Fluorescence resonance energy transfer |
| HMG-CoA | 3-hydroxy-3-methylglutaryl-coenzyme A |
| LCR | Ligase chain reaction |
| MASDA | Multiple allele specific diagnostic assay |
| NASBA | Nucleic acid sequence based amplification |
| OATP | Na+-independent organic anion transporting polypeptide |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

Table 1—Mutation Detection Techniques

General: DNA sequencing, Sequencing by hybridisation

Scanning: PTT*, SSCP, DGGE, TGGE, Cleavase, Heteroduplex analysis, CMC, Enzymatic mismatch cleavage

* Note: not useful for detection of promoter polymorphisms.

Hybridisation Based

Solid phase hybridisation: Dot blots, MASDA, Reverse dot blots, Oligonucleotide arrays (DNA Chips)

Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York)

Extension Based: ARMS™, ALEX™—European Patent No. EP 332435 B1 (Zencca Limited), COPS—Gibbs et al (1989), Nucleic Acids Research, 17, 2347.

Incorporation Based: Mini-sequencing, APEX

Restriction Enzyme Based: RFLP, Restriction site generating PCR

Ligation Based: OLA

Other: Invader assay

Table 2—Signal Generation or Detection Systems

Fluorescence: FRET, Fluorescence quenching, Fluorescence polarisation—United Kingdom Patent No. 2228998 (Zeneca Limited)

Other: Chemiluminescence, Electrochemiluminescence, Raman, Radioactivity, Colorimetric, Hybridisation protection assay, Mass spectrometry

Table 3—Further Amplification Methods

SSR, NASBA, LCR, SDA, b-DNA

Table 4—Protein variation detection methods

Immunoassay

Immunohistology

Peptide sequencing

Immunoassay techniques are known in the art e.g. A Practical Guide to ELISA by D M Kemeny, Pergamon Press 1991; Principles and Practice of Immunoassay, 2^{nd} edition, C P Price & D J Newman, 1997, published by Stockton Press in USA & Canada and by Macmillan Reference in the United Kingdom. Histological techniques are known in the art e.g. Theory and Practice of Histological Techniques, 4^{th} Edition, edited by J D Bancroft and A Stevens, Churchill Livingstone, 1996.

Preferred mutation detection techniques include ARMS™, ALEX™, COPS, Taqman, Molecular Beacons, RFLP, and restriction site based PCR and FRET techniques.

Particularly preferred methods include ARMS™ and RFLP based methods. ARMS™ is an especially preferred method.

In a further aspect, the methods of the invention are used to assess the pharmacogenetics of a drug transportable by OATPB.

Assays, for example reporter-based assays, may be devised to detect whether one or more of the above polymorphisms affect transcription levels and/or message stability.

Individuals who carry particular allelic variants of the OATPB gene may therefore exhibit differences in their ability to regulate protein biosynthesis under different physiological conditions and will display altered abilities to react to different diseases. In addition, differences arising as a result of allelic variation may have a direct effect on the response of an individual to drug therapy. The methods of the invention may be useful both to predict the clinical response to such agents and to determine therapeutic dose.

In a further aspect, the methods of the invention, are used to assess the predisposition and/or susceptibility of an individual to diseases mediated by OATPB. This may be particularly relevant in the development of hyperlipoproteinemia and cardiovascular disease and the present invention may be used to recognise individuals who are particularly at risk from developing these conditions.

In a further aspect, the methods of the invention are used in the development of new drug therapies which selectively target one or more allelic variants of the OATPB gene. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further aspect of the invention the presence or absence of variant nucleotides is detected by reference to the loss or gain of, optionally engineered, sites recognised by restriction enzymes.

According to another aspect of the present invention there is provided a human OATPB gene or its complementary strand comprising a variant allelic polymorphism at one or more of positions defined herein or a fragment thereof of at least 20 bases comprising at least one novel polymorphism.

Fragments are at least 17 bases, more preferably at least 20 bases, more preferably at least 30 bases.

According to another aspect of the present invention there is provided a polynucleotide comprising at least 20 contiguous bases of the human OATPB gene and comprising an allelic variant selected from:

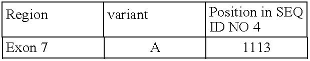

According to another aspect of the present invention there is provided a human OATPB gene or its complementary strand comprising a polymorphism, preferably corresponding with one or more the positions defined herein or a fragment thereof of at least 20 bases comprising at least one polymorphism.

Fragments are at least 17 bases, more preferably at least 20 bases, more preferably at least 30 bases.

The invention further provides a nucleotide primer which can detect a polymorphism of the invention.

According to another aspect of the present invention there is provided an allele specific primer capable of detecting a OATPB gene polymorphism, preferably at one or more of the positions as defined herein.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17- 50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting a OATPB gene polymorphism, preferably at one or more of the positions defined herein.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided an allele specific primer or an allele specific oligonucleotide probe capable of detecting a OATPB gene polymorphism at one of the positions defined herein.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

In another aspect of the invention, the single nucleotide polymorphisms of this invention may be used as genetic markers in linkage studies. This particularly applies to the polymorphisms of relatively high frequency. The OATPB gene is on chromosome 11. Low frequency polymorphisms may be particularly useful for haplotyping as described below. A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs. One approach to identifying mutations or polymorphisms which are correlated with clinical response is to carry out an association study using all the haplotypes that can be identified in the population of interest. The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine. Ann Hum Genet (1998) 62:481-90, De Stefano V, Dekou V, Nicaud V, Chasse J F, London J, Stansbie D, Humphries S E, and Gudnason V; and Variation at the von willebrand factor (vWF) gene locus is associated with plasma vWF:Ag levels: identification of three novel single nucleotide polymorphisms in the vWF gene promoter. Blood (1999) 93:4277-83, Keightley A M, Lam Y M, Brady J N, Cameron C L, Lillicrap D).

According to another aspect of the present invention there is provided a computer readable medium comprising at least one novel sequence of the invention stored on the medium. The computer readable medium may be used, for example, in homology searching, mapping, haplotyping, genotyping or pharmacogenetic analysis.

According to another aspect of the present invention there is provided a method of treating a human in need of treatment with a drug transportable by OATPB in which the method comprises:

i) detection of a polymorphism in OATPB in a human, which method comprises determining the sequence of the human at one of the following positions: position 1113 of SEQ ID NO: 4 and/or position 312 of SEQ ID NO 5; and

ii) administering an effective amount of the drug.

Preferably determination of the status of the human is clinically useful. Examples of clinical usefulness include deciding which statin drug or drugs to administer and/or in deciding on the effective amount of the statin drug or drugs. Statins already approved for use in humans include atorvastatin, cerivastatin, fluvastatin, pravastatin and simvastatin. The reader is referred to the following references for further information: Drugs and Therapy Perspectives (12 May 1997), 9: 1-6; Chong (1997) Pharmacotherapy 17: 1157-1177; Kellick (1997) Formulary 32: 352; Kathawala (1991) Medicinal Research Reviews. 11: 121-146; Jahng (1995) Drugs of the Future 20: 387-404, and Current Opinion in Lipidology, (1997), 8, 362-368. Another statin drug of note is compound 3a (S-4522) in Watanabe (1997) Bioorganic and Medicinal Chemistry 5: 437-444. The term “drug transportable by OATPB” means that transport by OATPB in humans is an important part of a drug exerting its pharmceutical effect in man. For example, some statins have to be transported to the liver by OATPC, which is highly homologous to OATPB, to exert their lipid lowering effects. Accordingly, OATPB is expected to be involved in statin transport.

According to another aspect of the present invention there is provided a method of treating a human in need of treatment with a drug transportable by OATPB in which the method comprises:

i) detection of a polymorphism in OATPB in a human, which method comprises determining the sequence of the human at one of the following positions: position 1113 of SEQ ID NO: 4 and/or position 312 of SEQ ID NO 5; and

ii) administering an effective amount of the drug.

According to another aspect of the present invention there is provided use of a drug transportable by OATPB in preparation of a medicament for treating a disease in a human determined as having a polymorphism defined herein. Preferably the disease is cardiovascular.

According to another aspect of the present invention there is provided a pharmaceutical pack comprising OATPB transportable drug and instructions for administration of the drug to humans tested for a polymorphism described therein, preferably at one or more of the positions defined herein.

According to another aspect of the present invention there is provided an allelic variant of human OATPB polypeptide comprising a glutamine at position 312 of SEQ ID NO 5 or a fragment thereof comprising at least 10 amino acids provided that the fragment comprises the allelic variant.

Fragments of polypeptide are at least 10 amino acids, more preferably at least 15 amino acids, more preferably at least 20 amino acids.

According to another aspect of the present invention there is provided an antibody specific for an allelic variant of human OATPB polypeptide as described herein.

Antibodies can be prepared using any suitable method. For example, purified polypeptide may be utilized to prepare specific antibodies. The term “antibodies” is meant to include polycional antibodies, monoclonal antibodies, and the various types of antibody constructs such as for example F(ab′)₂, Fab and single chain Fv. Antibodies are defined to be specifically binding if they bind the allelic variant of OATPB with a Kₐ of greater than or equal to about 10⁷ M⁻¹. Affinity of binding can be determined using conventional techniques for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011, 4,902,614, 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394 (1989).

Once isolated and purified, the antibodies may be used to detect the presence of antigen in a sample using established assay protocols, see for example “A Practical Guide to ELISA” by D. M. Kemeny, Pergamon Press, Oxford, England.

According to another aspect of the invention there is provided a diagnostic kit comprising an antibody of the invention.

The invention will now be illustrated but not limited by reference to the following Examples. All temperatures are in degrees Celsius.

In the Examples below, unless otherwise stated, the following methodology and materials have been applied.

AMPLITAQ™, available from Perkin-Elmer Cetus, is used as the source of thermostable DNA polymerase.

General molecular biology procedures can be followed from any of the methods described in “Molecular Cloning—A Laboratory Manual” Second Edition, Sambrook, Fritsch and Maniatis (Cold Spring Harbor Laboratory, 1989) or in “Current Protocols in Molecular Biology Volumes 1-3, edited by F M Asubel, R Brent and R E Kingston; published by John Wiley, 1998.

Electropherograms were obtained in a standard manner: data was collected by ABI377 data collection software and the wave form generated by ABI Prism sequencing analysis (2.1.2).

### EXAMPLE 1

Identification of Polymorphisms

1. Methods

The RNA sequence of OATPB (AB026256) was used to design PCR primers to amplify the full length of the OATPB sequence in approximately 400 base pair overlapping regions. Fifteen individual liver cDNA preparations were used as templates for PCR amplification. The products were then sequenced by dye-primer sequencing in the forward and the reverse direction. The alignment of sequence traces enabled the identification of polymorphisms. The frequency of the polymorphisms was confirmed by primer extension analysis by HPLC (WAVE) method using genomic DNA from 23 individuals and by sequencing.

PCR products

| | | | Primer used |
|---|---|---|---|
| | PCR forward | PCR reverse | for confirmation |
| Polymorphism | oligo | oligo | of polymorphism |
| 1113 (G/A) | (SEQ ID NO: 1) | (SEQ ID NO: 2) | (SEQ ID NO: 3) |

PCR Conditions:

18 μl ABgene 2 mM Reddy load

1 μl 5 μM primer pair mix

1 μl genomic DNA (various individuals)

PCR Programme:

94° C. 1 min; (94° C. 30 sec, 58° C. 30 sec, 72° C. 2 min) for 34 cycles; 72° C. 10 min.

2 Polymorphism

| Position of | | | | | Frequency |
|---|---|---|---|---|---|
| SEQ ID | | | | Protein sequence | variant |
| NO 4 | Exon | Variation | Codon | SEQ ID NO 5 | allele |
| 1113 | 7 | G/A | cga-caa | Arg312Gln | 12% A |

1 A method for the detection of a polymorphism in OATPB in a human which method comprises determining the sequence of the human at one of the following positions: position 1113 of SEQ ID NO: 4 and/or position 312 of SEQ ID NO 5.

2 A method according to claim 1 wherein the polymorphism at position 1113 is presence of G and/or A and the polymorphism at position 312 is presence of Arg and/or Gln.

3 A method according to claim 1 or 2 wherein the method for detection of a nucleic acid polymorphism is selected from amplification refractory mutation system and restriction fragment length polymorphism.

4 Use of a method defined in any of claims 1-3 to assess the pharmacogenetics of a drug transportable by OATPB.

5 A polynucleotide comprising at least 20 contiguous bases of the human OATPB gene and comprising an allelic variant which is:

| | | Position in SEQ |
|---|---|---|
| Region | variant | ID NO 4 |
| Exon 7 | A | 1113 |

6 an allele specific primer capable of detecting a OATPB gene polymorphism at position 1113 in SEQ ID NO: 4.

7 An allele specific oligonucleotide probe capable of detecting a OATPB gene polymorphism at position 1113 in SEQ ID NO: 4.

8 A diagnostic kit comprising an allele specific oligonucleotide probe of claim 7 and/or an allele-specific primer of claim 6.

9 Use of a drug transportable by OATPB in preparation of a medicament for treating a disease in a human determined as having a polymorphism at one of the following positions: position 1113 of SEQ ID NO: 4 and/or position 312 of SEQ ID NO 5.

10 An allelic variant of human OATPB polypeptide comprising a glutamine at position 312 of SEQ ID NO 5 or a fragment thereof comprising at least 10 amino acids provided that the fragment comprises the allelic variant.

11 An antibody specific for an allelic variant of human OATPB polypeptide as described herein.

12 A diagnostic kit comprising an antibody of claim 11.


SEQUENCE LISTING (5 sequences)

SEQ ID NO: 1 - DNA, 20 bp, Homo sapiens
cagggtcacacaggacacac

SEQ ID NO: 2 - DNA, 20 bp, Homo sapiens
cccagccgaagtgtaatgtt

SEQ ID NO: 3 - DNA, 20 bp, Homo sapiens
acgtgagcttcagtttcggc

SEQ ID NO: 4 - DNA, 2354 bp, Homo sapiens
ctgtgtggcccaagaagaactgaccccgtgtctggagctcccaccgttattgcatccctgctgtggctcacctgctgctgtctccaggagcccctgagaagatttgcctcctctcccctgctaagctccaggtcctgagattgaattaggggctggagctcactgcactccagcagtcatgggacccaggatagggccagcgggtgaggtaccccaggtaccagacaaggaaaccaaagccacaatgggcacagaaaacacacctggaggcaaagccagcccagaccctcaggacgtgcggccaagtgtgttccataacatcaagctgttcgttctgtgccacagcctgctgcagctggcgcagctcatgatctccggctacctaaagagctccatctccacagtggagaagcgcttcggcctctccagccagacgtcggggctgctggcctccttcaacgaggtggggaacacagccttgattgtgtttgtgagctattttggcagccgggtgcaccgaccccgaatgattggctatggggctatccttgtggccctggcgggcctgctcatgactctcccgcacttcatctcggagccataccgctacgacaacaccagccctgaggatatgccacaggacttcaaggcttccctgtgcctgcccacaacctcggccccagcctcggccccctccaatggcaactgctcaagctacacagaaacccagcatctgagtgtggtggggatcatgttcgtggcacagaccctgctgggcgtgggcggggtgcccattcagccctttggcatctcctacatcgatgactttgcccacaacagcaactcgcccctctacctcgggatcctgtttgcagtgaccatgatggggccaggcctggcctttgggctgggcagcctcatgctgcgcctttatgtggacattaaccagatgccagaaggtggtatcagcctgaccataaaggacccccgatgggtgggtgcctggtggctgggtttcctcatcgctgccggtgcagtggccctggctgccatcccctacttcttcttccccaaggaaatgcccaaggaaaaacgtgagcttcagtttcggcgaaaggtcttagcagtcacagactcacctgccaggaagggcaaggactctccctctaagcagagccctggggagtccacgaagaagcaggatggcctagtccagattgcaccaaacctgactgtgatccagttcattaaagtcttccccagggtgctgctgcagaccctacgccaccccatcttcctgctggtggtcctgtcccaggtatgcttgtcatccatggctgcgggcatggccaccttcctgcccaagttcctggagcgccagttttccatcacagcctcctacgccaacctgctcatcggctgcctctccttcccttcggtcatcgtgggcatcgtggtgggtggcgtcctggtcaagcggctccacctgggccctgtgggatgcggtgccctttgcctgctggggatgctgctgtgcctcttcttcagcctgccgctcttctttatcggctgctccagccaccagattgcgggcatcacacaccagaccagtgcccaccctgggctggagctgtctccaagctgcatggaggcctgctcctgcccattggacggctttaaccctgtctgcgaccccagcactcgtgtggaatacatcacaccctgccacgcaggctgctcaagctgggtggtccaggatgctctggacaacagccaggttttctacaccaactgcagctgcgtggtggagggcaaccccgtgctggcaggatcctgcgactcaacgtgcagccatctggtggtgcccttcctgctcctggtcagcctgggctcggccctggcctgtctcacccacacaccctccttcatgctcatcctaagaggagtgaagaaagaagacaagactttggctgtgggcatccagttcatgttcctgaggattttggcctggatgcccagccccgtgatccacggcagcgccatcgacaccacctgtgtgcactgggccctgagctgtgggcgtcgagctgtctgtcgctactacaataatgacctgctccgaaaccggttcatcggcctccagttcttcttcaaaacaggttctgtgatctgcttcgccttagttttggctgtcctgaggcagcaggacaaagaggcaaggaccaaagagagcagatccagccctgccgtagagcagcaattgctagtgtcggggccagggaagaagccagaggattcccgagtgtgagctgtcttggggccccacctggccaagagtagcagccacagcagta

SEQ ID NO: 5 - protein, 709 aa, Homo sapiens
MGPRIGPAGEVPQVPDKETKATMGTENTPGGKASPDPQDVRPSVFHNIKLFVLCHSLLQLAQLMISGYLKSSISTVEKRFGLSSQTSGLLASFNEVGNTALIVFVSYFGSRVHRPRMIGYGAILVALAGLLMTLPHFISEPYRYDNTSPEDMPQDFKASLCLPTTSAPASAPSNGNCSSYTETQHLSVVGIMFVAQTLLGVGGVPIQPFGISYIDDFAHNSNSPLYLGILFAVTMMGPGLAFGLGSLMLRLYVDINQMPEGGISLTIKDPRWVGAWWLGFLIAAGAVALAAIPYFFFPKEMPKEKRELQFRRKVLAVTDSPARKGKDSPSKQSPGESTKKQDGLVQIAPNLTVIQFIKVFPRVLLQTLRHPIFLLVVLSQVCLSSMAAGMATFLPKFLERQFSITASYANLLIGCLSFPSVIVGIVVGGVLVKRLHLGPVGCGALCLLGMLLCLFFSLPLFFIGCSSHQIAGITHQTSAHPGLELSPSCMEACSCPLDGFNPVCDPSTRVEYITPCHAGCSSWVVQDALDNSQVFYTNCSCVVEGNPVLAGSCDSTCSHLVVPFLLLVSLGSALACLTHTPSFMLILRGVKKEDKTLAVGIQFMFLRILAWMPSPVIHGSAIDTTCVHWALSCGRRAVCRYYNNDLLRNRFIGLQFFFKTGSVICFALVLAVLRQQDKEARTKESRSSPAVEQQLLVSGPGKKPEDSRV